(19) United States
(12) Patent Application Publication
(10) Pub. No.: US 2006/0027539 A1
(43) Pub. Date: Feb. 9, 2006

(54) Non-thermal plasma generator device

(76) Inventor: Czeslaw Golkowski, Ithaca, NY (US)

Correspondence Address: BROWN & MICHAELS, PC;400 M & T BANK BUILDING, 118 NORTH TIOGA ST, ITHACA, NY, 14850, US

(21) Appl. No.: 11/240,032

(22) Filed: Sep. 30, 2005

Related U.S. Application Data
(63) Continuation-in-part of application No. 10/429,542, filed on May 2, 2003.

(51) Int. Cl. B23K 9/00

(52) U.S. Cl. 219/121.59; 219/121.48; 219/121.36; 422/23

(57) ABSTRACT

A non-thermal plasma generation device for non-destructively decontaminating sensitive surfaces, such as living tissue, electronic equipment and other surfaces that cannot tolerate high temperatures or aggressive chemicals, using the free radicals and excited states of gas produced in an atmospheric-pressure air plasma. The plasma is preferably generated by a stable, self-igniting discharge in a resonant waveguide system, driven by a magnetron or other high power microwave source, operating in a pulsed mode.

### REFERENCE TO RELATED APPLICATIONS

This is a continuation-in-part patent application of copending application Ser. No. 10/429,542, filed May 2, 2003, entitled “Non-thermal plasma generator device”. The aforementioned application(s) are hereby incorporated herein by reference.

### BACKGROUND OF THE INVENTION

1. Field of the Invention

The present invention relates generally to non-thermal plasma generating devices, and more particularly to such devices used to decontaminate sensitive surfaces, such as living tissue, electronic equipment and other surfaces which cannot tolerate high temperature or aggressive chemicals.

2. Description of Related Art

Bacteria, chemicals, and other harmful organisms contaminate sensitive surfaces, such as living tissue, food products, and other surfaces that humans come into contact on a daily basis. It is necessary to decontaminate these surfaces to prevent illness from affecting the humans who may come into contact with the contaminated surface. A common manner of performing the decontamination is to expose the surface to a plasma generated from an inert gas, such as argon, or expose the surface to radiation. While such systems are effective, they are also costly to operate and could adversely affect the surface being treated.

Plasmas have been used in a wide variety of industrial application, such as semiconductor fabrication and coatings of reflective films for window panels and compact discs. The principal advantage of plasma cleaning is that it is an “all dry” process, generates minimal effluent, does not require hazardous pressures, and is applicable to a wide variety of vacuum compatible materials, including silicon, metals, glass, and ceramics.

Labat, U.S. Pat. No. 4,924,061, has conducting conduits (with terminal elements 31, 32) together with the sleeve (terminal element 30) form a coaxial wave-guide for the microwaves. The arrangement presented in FIG. 1 creates a tunable (notice sliding parts) resonant coaxial cavity closed on end by the piston 9 and the open end with maximum of the electric field on the opposite side (terminal elements 30, 31, 32), where the plasma is generated. In our case we do not have any coaxial wave-guide or coaxial resonant cavity.

Griffiths, et al, U.S. Pat. No. 5,503,807 creates plasma for gas sterilization. The plasma is created in an “arc mode”, between elements 27 and 27′ (see FIG. 2). In contrast, the present invention creates plasma in a “glow mode” at a single element.

Another Griffths, et. al., patent, No. 5,512,244, routes the gas through a continuous tube passing through the resonator, rather than passing the gas into the resonator as is done in the present invention.

U.S. Pat. No. 5,961,772 to Selwyn describes an atmospheric, non-thermal plasma jet which produces metastable and reactive species that are useful for etching and cleaning surfaces. The Background section of the '772 patent describes the benefits of and the state of the art in non-thermal, atmospheric plasmas in great detail, and with the exception of that invention's objects and advantages, its background section is hereby incorporated by reference.

Moisan, U.S. Pat. No. 6,298,806, is a device in which plasma is initiated by a surface-wave supported by a dielectric inserted into a wave-guide (applicator). The dielectric is inserted into the wave-guide where the power coupling is maximized, i.e. at the maximum of the electric field. In Moisan device the dielectric plays a crucial role of generating plasma near the dielectric surface and therefore the plasma is in direct contact with the dielectric. In our invention the dielectric (quartz) inner tube is used only as gas conduit and as a support for the conducting ring. The plasma in our device is generated by the ring inside the wave-guide and inside the outer dielectric (quartz) tube. The role of the outer tube is to prevent the gas spreading into the cavity. In Moisan device the tubes have to be extended above the applicator in order to support the surface wave. In the present invention, it is desireable to limit surface wave generation.

### SUMMARY OF THE INVENTION

The present invention provides a plasma generation device for non-destructive decontamination of sensitive surfaces, comprising a waveguide of predetermined length extending between first and second opposite ends and including a cavity in which waves may propagate; an electromagnetic wave generator connected to the waveguide, adjacent its first end, for generating waves of electromagnetic energy having predetermined wavelengths in the waveguide; a gas conduit introducing gas into the cavity, and a gas exit having a “glow mode” plasma-generating ring at a predetermined distance from the second end where the electric field is particularly strong, and an exit port leading from the plasma-forming ring, through which the plasma of metastable and excited state of gas may flow. A sensitive surface a predetermined distance beneath the exit port will be impacted by the plasma which, in turn, will kill certain types of bacteria present thereon.

### BRIEF DESCRIPTION OF THE DRAWING

FIG. 1 is a perspective view of a first embodiment of the present invention with a portion broken away;

FIG. 2 is an exploded perspective view of the gas introduction and plasma generation system of a first embodiment of the present invention;

FIG. 3 is a cross-sectional view taken along line 3-3 of FIG. 1; and

FIG. 4 is a diagrammatic sectional view of a first embodiment of the present invention.

FIG. 5 is a diagrammatic sectional view of a second embodiment of the present invention

### DETAILED DESCRIPTION OF THE INVENTION

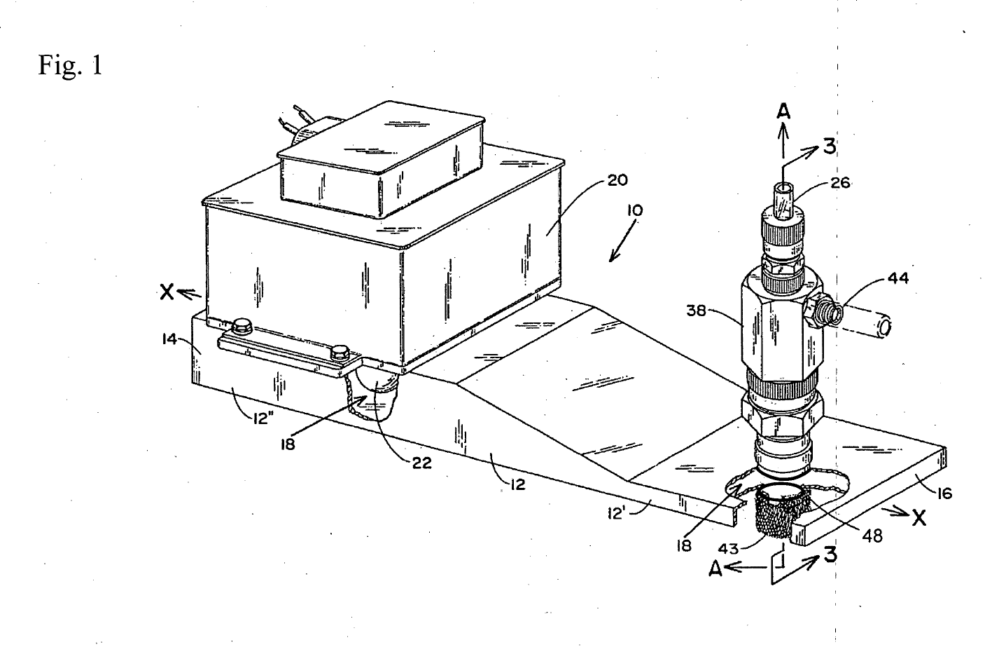

Referring now to the drawings, in which like reference numerals refer to like parts throughout, there is seen in FIG. 1 a plasma generation device, designated generally by reference numeral 10, for non-destructively decontaminating sensitive surfaces, such as living tissue, electronic equipment and other surfaces that cannot tolerate high temperatures or aggressive chemicals, using the free radicals and excited states of gas produced in an atmospheric-pressure air plasma. As will be described in greater detail hereinafter, the plasma is preferably generated by a stable, self-igniting discharge in a resonant waveguide system, driven by a magnetron or other high power microwave source, operating in a pulsed mode.

Plasma generation device 10 generally comprises a waveguide 12 extending a predetermined length L between closed, opposing ends 14, 16 along a longitudinal axis X-X, and defining a resonant cavity 18 in which waves may propagate. The length L will usually be an even multiple of ¼λ, where λ is the wavelength of the waves. The waves are generated by a magnetron (or equivalent high energy power microwave source capable of producing pulsating electromagnetic waves) 20, which has its electromagnetic wave generating portion 22 positioned within cavity 18 adjacent the first end 14.

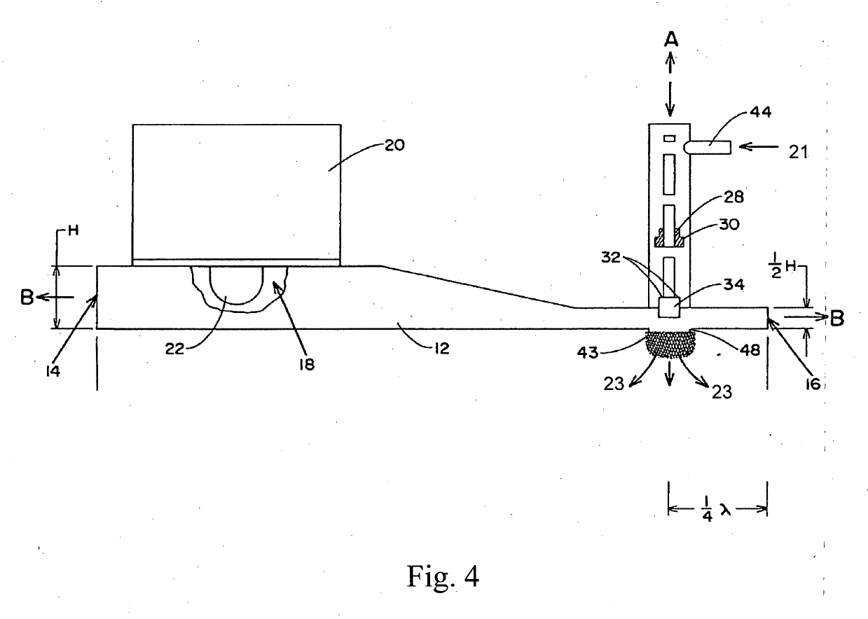

Gas is introduced through a gas introduction system, designated generally by reference numeral 24, positioned in fluid communication with cavity 18 and located a predetermined distance from second end 16, preferably ¼λ or an odd multiple of ¼λ, where the wave energy in the waveguide is at a maximum (see FIG. 4). Plasma is created by a plasma generating conductive field concentrator 34 in the gas introduction system, and is discharged through port 48 and, optionally, grid filter 43.

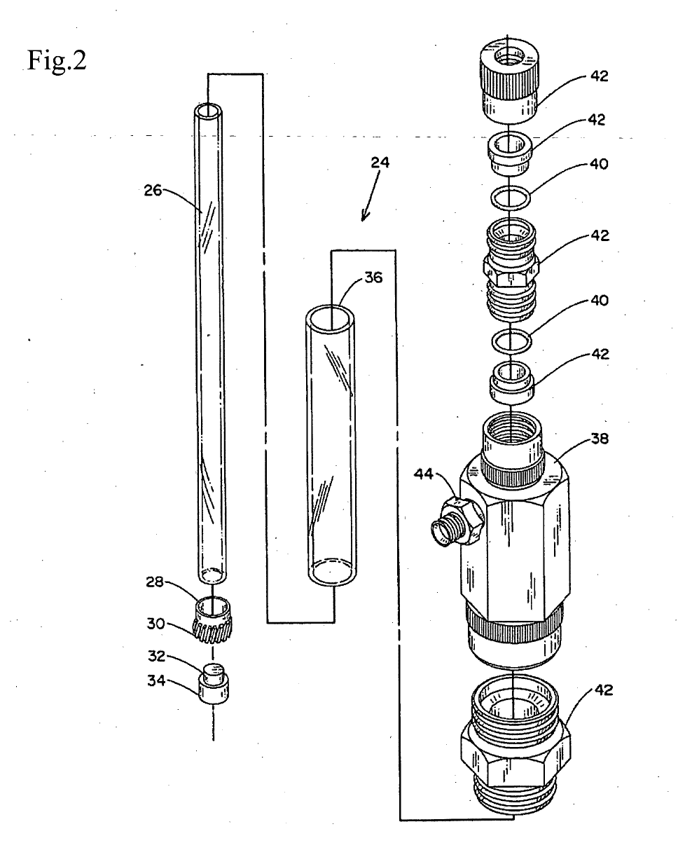

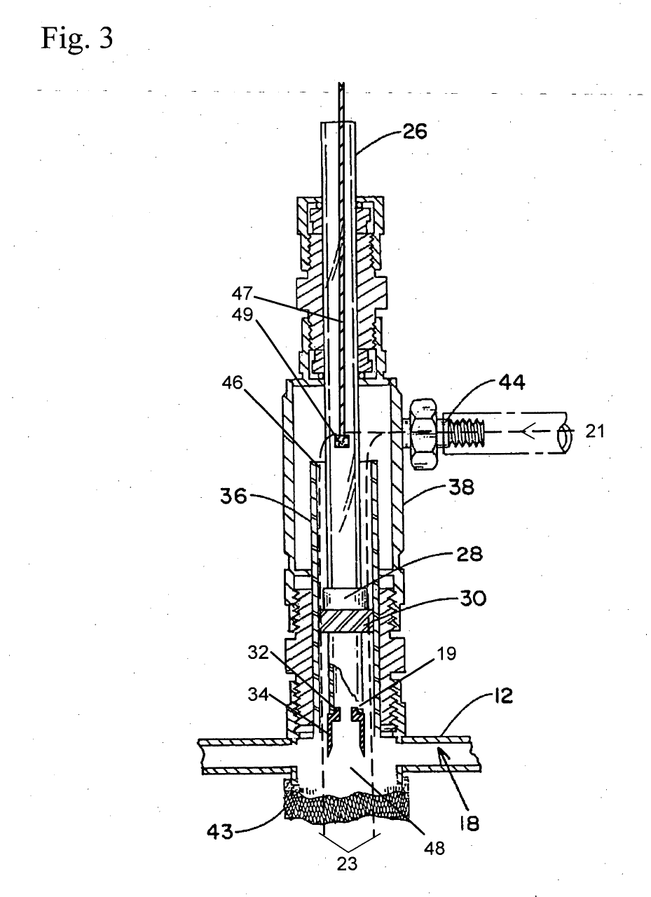

Referring to FIGS. 2 and 3, gas introduction system 24 essentially comprises an inner tube 26 that is used to support a ring 28 that includes a plurality of vanes 30 formed on its outer surface and a conductive field concentrator 32 having a lower portion 34 that is preferably terminates in a sharp edge. The location of the field concentrator just inside the waveguide at a point of maximum energy causes a plasma to form at the sharp edge of the field concentrator.

An outer tube 36 is concentrically positioned in spaced relation around inner tube 26. Ring 28 and vanes 30 are positioned between inner tube 26 and outer tube 36. As can be seen, outer tube 36 is shorter than inner tube 26, which permits gas to enter and pass between the two tubes.

Gas introduction system 24 further comprises a housing 38 that surrounds and supports the tubes 26 and 36, and a series of sealing rings 40 and tube supports 42 that maintain inner tube 26 in sealed, concentric position within housing 38.

Tubes 26 and 36 co-axially extend along a longitudinal axis A-A that is essentially transverse to the longitudinal axis X-X along which waveguide 12 extends.

In operation, compressed air or other gas mixture 21 is introduced into gas introduction system 24 through tube 44, and passes into the area separating inner tube 26 and outer tube 36, as indicated by arrow 46. If desired, atomized and/or vaporized substances such as hydrogen peroxide or bleach may be introduced into the airstream for additional sterilizing capability.

Optionally, gas or substances to be introduced may be injected through the center of tube 26, if one or more ports 19 are provided through which they may mix with the main gas stream 46.

The air flows downwardly and is mixed into a turbulent state by passing through vanes 30 and into the plasma at the field concentrator 34. This produces a plasma stream 23 that exits through a port 48 formed through the bottom wall of waveguide 12. The system may further include a conductive mesh 43 at its outlet. Mesh 43 prevents any dissipation of microwaves.

The effluent of metastable and excited states of air/gas molecules and whatever additives may have been introduced extends a few centimeters away from port 48, although its maximum effects are present in about the first centimeter of discharge.

In most cases, the plasma discharge from the field concentrator 34 will begin as soon as the energy field is established in the waveguide 12. However, on the rare instances when the plasma does not immediately self-start, a small piece of Silicon Carbide 49, supported on a dielectric rod 47, may be inserted through the hollow center of inner tube 26. As the Silicon Carbide piece approaches the electric field around the field concentrator 34, a plasma will begin to be generated, and the Silicon Carbide may then be withdrawn.

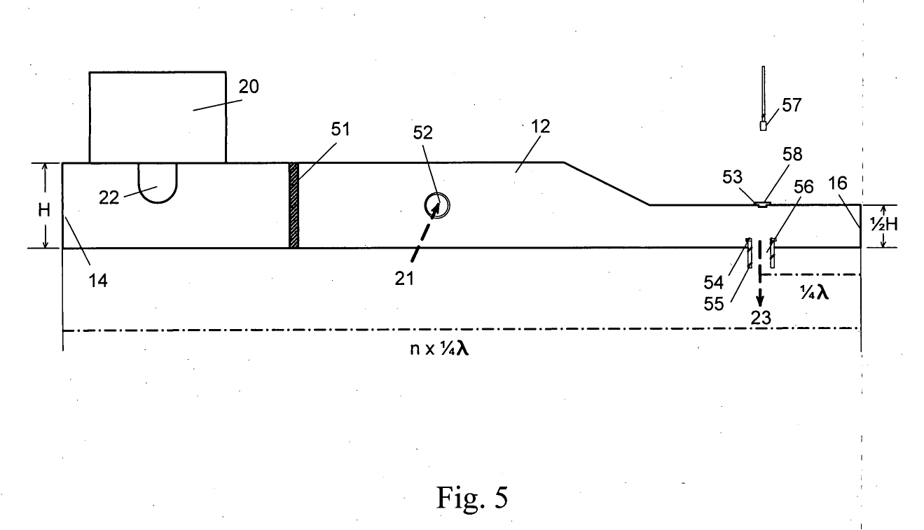

FIG. 5 shows a second embodiment of the invention, which does not use the gas introduction system 24 of the first embodiment. Instead, gas 21 is introduced into the waveguide 12 through a port 52. Optionally, a dielectric gas barrier 51 may be provided between port 52 and generator 22, in order to keep the gas 21 from the microwave generator and minimize leakage problems. The barrier 51 is preferably made of PTFE (Teflon®), but could be any appropriate material which is transparent to microwaves but gas impermeable.

The field concentrator 56 is in the form of a conductive ring having sharp edges, mounted, as in the first embodiment, a predetermined distance from second end 16, preferably ¼λ or an odd multiple of ¼λ, where the wave energy in the waveguide is at a maximum. The field concentrator 56 is supported by a support tube 54, which can be made of a dielectric material. Alternatively, the support tube 54 could be of a conductive material, in which case the field concentrator 56 might be formed as a part of the support tube 54 or as a separate piece. Plasma is created by the field concentrator 56, and exits with the gas stream 23 through discharge port 55. If desired, a mesh filter as used in the first embodiment (not shown), or a flexible tube for routing the stream (not shown) may be attached to the discharge port 55.

A small hole 53, with a removable cover 58, may be provided in the top of the waveguide immediately above the field concentrator 56. As noted in the first embodiment, if a plasma does not immediately start when the field is turned on, a small piece of Silicon Carbide 57 may be introduced through the hole 53 and into proximity of the field concentrator 56 to trigger the plasma. When the plasma has started, the Silicon Carbide 57 is withdrawn, and the hole 53 closed by cover 58 so that gas does not leak out the top of the waveguide.

### EXAMPLE 1

### Destruction of Volatile Organic Compounds (Voc) in Air With Varying Pulse Width Within a Range of 1-2.5 Microsecond and Repetition Rate 20,000 Pps.

Recent tests utilizing the above described device have shown the following results:

The removal of 100% of propane in air was achieved for the flow rate of 60 slm (standard liters per minute) with the following output gas temperature:
temp. ˜80° C., 100% removal up to 1,000 ppm (parts per million)
temp. ˜70° C., 100% removal up to 500 ppm
temp. ˜50° C., 100% removal up to ˜10 ppm

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A plasma generation device for non-destructive decontamination of sensitive surfaces, comprising:
a) an electromagnetic wave generator for generating pulsating waves of electromagnetic energy having a wavelength;
b) a waveguide having a first closed end and a second closed end, and a length therebetween, the length being an even multiple of one-quarter of the wavelength, defining a cavity in which waves may propagate, the wave generator being connected to the waveguide adjacent its said first end;
c) a gas conduit in fluid communication with the cavity for introduction of a flow of gas into the cavity;
d) a conductive field concentrator having at least one sharp edge, located in the cavity at a point of maximum energy in the cavity; and
e) an exit port in fluid communication with the cavity, adjacent the field concentrator, through which plasma and gas flows out of the cavity.

2. The plasma generation device of claim 1, wherein said waveguide is rectangular in cross-section.

3. The plasma generation device of claim 1, in which the point of maximum energy is at a distance from the second end of the cavity which is an odd multiple of one-quarter wavelength.

4. The plasma generation device of claim 3, in which the odd multiple is one-quarter.

5. The plasma generation device of claim 1, wherein said electromagnetic wave generator is a pulsed magnetron.

6. The plasma generation device of claim 1, wherein the gas conduit comprises:
an inner tube of dielectric material having an upper end and a lower end and a length therebetween;
an outer tube of dielectric material having an upper end and a lower end and a length therebetween which is less than the length of the inner tube, mounted coaxial with the inner tube and surrounding a lower portion thereof, such that the lower end of the inner tube and the lower end of the outer tube are adjacent to each other;
a gas inlet in fluid communication with the upper end of the outer tube;
the conductive field concentrator being mounted adjacent the lower ends of the inner tube and the outer tube;
such that gas introduced into the gas inlet flows between the inner tube and the outer tube, past the conductive field concentrator, and into the cavity.

7. The plasma generation device of claim 6, further comprising a ring of vanes located between the inner tube and the outer tube, adjacent to the lower ends of the tubes.

8. The plasma generation device of claim 1, in which the conductive field concentrator comprises a conductive ring positioned adjacent to the exit port, such that gas introduced into the cavity through the inlet port flows through the conductive ring and out the exit port.

9. The plasma generation device of claim 1, further comprising a conductive mesh covering the exit port.

10. The plasma generation device of claim 1, in which the cavity is divided between the wave generator and the gas inlet port by a barrier which is transparent to radio and microwaves and impermeable to gas.

11. A method of creating a plasma for non-destructive decontamination of sensitive surfaces using a plasma generator comprising an electromagnetic wave generator for generating pulsating waves of electromagnetic energy having a wavelength; waveguide having a first closed end and a second closed end, and a length therebetween, the length being an even multiple of one quarter of the wavelength, defining a cavity in which waves may propagate, the wave generator being connected to the waveguide adjacent its said first end; a gas conduit in fluid communication with the cavity for introduction of a flow of gas into the cavity; conductive field concentrator having at least one sharp edge, located in the cavity at a point of maximum energy in the cavity; and an exit port in fluid communication with the cavity, adjacent the field concentrator, through which plasma and gas flows out of the cavity; comprising the steps of:
generating electromagnetic energy in the cavity with the electromagnetic wave generator;
introducing a gas stream into the cavity through the gas conduit;
withdrawing plasma from the exit port.

12. The method of claim 11, further comprising the step of introducing an additional atomized and/or vaporized fluid into the gas stream.

13. The method of claim 12, in which the fluid is hydrogen peroxide.

14. The method of claim 12, in which the fluid is bleach.

15. The method of claim 11, further comprising the step of triggering a plasma at the conducting field concentrator by bringing a piece of silicon carbide into proximity of the field concentrator until a plasma starts.

16. The method of claim 11, further comprising the step of applying the plasma from the exit port to a material.
